(19) United States
(12) Patent Application Publication
(10) Pub. No.: US 2020/0085381 A1
(43) Pub. Date: Mar. 19, 2020

(54) Method and apparatus for detecting a needle in magnetic-resonance images

(71) Applicant: Siemens Healthcare GmbH, Erlangen (DE)

(72) Inventors: Jonathan Weine, Bochum (DE); Florian Maier, Buckenhof (DE)

(21) Appl. No.: 16/570,079

(22) Filed: Sep. 13, 2019

(30) Foreign Application Priority Data
Sep. 13, 2018 (DE) 10 2018 215 618.2

(51) Int. Cl. A61B 5/00; G01R 33/28; G01R 33/483; G01R 33/56; G06T 7/70; G06K 9/78; A61B 5/055; G06N 3/04; G06N 3/08

(52) U.S. Cl. CPC A61B 5/7267; G01R 33/286; G01R 33/4835; G01R 33/5608; G06T 7/70; G06K 9/78; G06K 2209/057; A61B 5/7271; G06N 3/0445; G06N 3/08; G06T 2207/10088; G06T 2207/20081; G06T 2207/20084; A61B 5/055

(57) ABSTRACT

A method for detecting a needle in magnetic-resonance images, wherein a needle artifact and/or a needle location is identified by means of algorithms based on artificial intelligence within the context of machine learning. Also, corresponding apparatus, control facility, and magnetic resonance tomography system.

### TECHNICAL FIELD

The disclosure relates to a method and an apparatus for detecting a needle in magnetic-resonance images.

### BACKGROUND

Nowadays, many medical interventions are performed with minimally invasive surgery. These methods also include, for example, needle biopsies or interventional applications as thermal ablations. Herein, the positioning of the devices used is very important since, for example, inaccurate tissue removal during a biopsy can result in the wrong diagnosis.

It is advisable to use a magnetic resonance scanner to monitor and guide the percutaneous placement of applicators or needles because of the good soft-tissue contrast and the absence of ionizing radiation. Online monitoring by means of magnetic resonance (“MR”) is typically performed with high-speed 2D slice images in which the, generally metallic, needles or applicators can be localized by the artifacts they generate. The positioning of the slices is decisive for the monitoring. This is because, in order to estimate the location of the needle, an interventional radiologist requires images that should contain the needle as completely as possible.

The degrees of freedom of the needle guidance and patient movement can have the effect that the tip of the needle is no longer present in the imaged slice without this being clearly evident from the image. Moreover, the user determines the position by estimation with the naked eye and hence this is error-prone and the estimated needle position can differ from person to person.

Herein, a further difficulty entails the fact that the central axis of the artifact in the image does not necessarily coincide with the location of the axis of symmetry of the needle since a shift occurs in dependence on the alignment relative to the BO field. The same applies to the tip of the artifact and the tip of the needle. The magnitude of the shift can be up to several millimeters.

Thus, there are two parts to the problem: on the one hand, the user must recognize the need for slice correction and, on the other, slice tracking is performed manually as a result of which the intervention takes longer and usually requires additional staff.

The determination of the exact position of the needle tip with correct slice alignment is problematic, but very important in order to ensure high-quality treatment.

To date, needle tracking has been performed using template matching algorithms. This generally works comparatively well in phantoms. However, in practical use, the method is insufficiently robust because of the variety of tissue types present (for example subcutaneous fat, muscle, liver, ribs and various transitional regions).

U.S. Pat. No. 8,526,691 describes the use of additional MR-visible markers for slice positioning. However, these lead to the problem that they complicate handling of the needle, because, for example, they reduce penetration depth.

### SUMMARY

It is an object of the present disclosure to provide an alternative, more comfortable method and a corresponding apparatus as well as a control facility for controlling a medical imaging system for generating image data with which the above-described drawbacks are avoided.

This object is achieved by a method, an apparatus, by a control facility, and a magnetic resonance tomography system as claimed.

With the method according to the disclosure for detecting a needle in magnetic-resonance images, a needle artifact and/or a needle location in magnetic-resonance images (from imaged slices) is identified by means of (trained) algorithms based on artificial intelligence within the context of machine learning, in particular by means of a deep-learning algorithm. Herein, preferably, needle artifacts are identified by means of a needle-artifact algorithm trained on needle artifacts and/or the needle location is identified by means of a needle-location algorithm trained on an estimation of the needle location. The slices are images taken by a magnetic resonance tomography system and include a number of picture elements. These picture elements can basically be understood to be pixels as they are a part of digital image data. However, pixel groups or computer-intelligible objects can also be considered to be picture elements. The slices can be present in the form of reconstructed images or in the form of raw data that is determined solely by the nature of the algorithm training. For all slices in a method step, it is assumed that they are not identical but at least adjacent and optimally spaced apart from one another. The thickness of a slice is normally between 5-10 mm. Thicker slices can be imaged slightly more quickly and with a better signal-to-noise ratio.

The method includes the following steps:

Location of Tracking Slices

First, location data on the location of a number of tracking slices is provided. Within the context of the disclosure, “location” should be understood to mean a position and an alignment, i.e. a location in space. For example, the location data can only include data on a plane in space, i.e. for example position vector and normal vector, position vector and two plane vectors or also three points in space. However, the location data can also directly include control data for imaging of a clearly defined slice for a magnetic resonance tomography system.

Theoretically, the method can be performed with one single tracking slice, at least as long as degrees of freedom of the needle alignment caused by other circumstances are known (if, for example, a point on the needle in the space outside the tracking slice is known). It is however preferable for the method to be performed with at least two tracking slices. Each further tracking slice improves the accuracy of the method, wherein obviously consideration should be paid to the time required to produce and evaluate the tracking slices. In particular, tracking slices improve the localization of the needle to a significant degree in uncertain regions (areas where it is uncertain where the needle could be located).

Initially, the needle position is estimated, for example based on general assumptions or optical measurements relating to the location of the needle. The tracking slices should lie orthogonally to an assumed needle alignment so that the assumed needle alignment depicts the surface normal.

Imaging the Tracking Slices

A number of tracking slices in the form of magnetic-resonance images are imaged with the location data provided. Herein, optimally, all tracking slices for which location data is available are imaged. The minimum is one tracking slice, but preferably there are two or more tracking slices.

These tracking slices are preferably imaged in the form of contrasts. It is not necessarily only one single contrast that is imaged within a tracking slice. Here, imaging of two or more contrasts is advantageous since this enables a better determination of the needle location. Hereinafter, the term “tracking slice” generally means the imaging of one or more contrasts. The picture elements of a tracking slice are generally pixels of these contrast images.

Identification of the Needle Artifacts in the Slice Images

The needle generates artifacts in the contrasts; here, these are referred to as “needle artifacts”. These needle artifacts are sought in the contrasts. In this step, the picture elements (pixels) in a tracking slice (or all the imaged tracking slices) which depict needle artifacts are identified. Therefore, an attempt is made to identify a map of the needle in the contrasts from the artifacts.

The identification is performed by means of a needle-artifact algorithm trained on needle artifacts. It should be noted in advance of the following description that it is theoretically possible to use the same needle-artifact algorithm to identify the needle artifacts in the tracking slice and in the monitoring slice described below. Since, however, the needle artifacts in the tracking slice and the monitoring slice generally differ systematically (in the case of tracking slices, they are generally punctiform or butterfly-shaped, in the case of monitoring slices, they generally have an oblong, straight shape), it is preferable always to use a specially trained needle-artifact algorithm for each tracking slice and monitoring slice.

These two algorithms can have the same systematic structure with only the training being performed differently. Therefore, it may be stated at this point that the identification is performed by means of a needle-artifact algorithm trained on needle artifacts in the tracking slice. This needle-artifact algorithm is preferably a machine-learning algorithm that has been trained on the recognition of needle artifacts. Such an algorithm is known to the person skilled in the art (for example, as will be explained in more detail below under the term U-net convolutional network).

Preferably, the artifact algorithm is used to produce a probability map containing information on the needle artifacts. This probability map particularly preferably has the same format as the relevant contrasts (same number of pixels), wherein this probability map contains the needle artifacts, or values for their probability, as picture elements in and in particular this is the only information in this probability map. A probability map is preferably based on needle-artifact segmentation. Therefore, the output value supplied by the algorithm is a probability map for the probability of a pixel of the tracking slice belonging to the needle.

Identification of a Needle Location

The location of the needle is identified by means of a needle-location algorithm trained on an estimation of the needle location. This identification is based on the previously identified picture elements which depict needle artifacts (i.e. generally on a probability map created from the pixels of a contrast). Theoretically, this would require only one tracking slice (as long as additional information on the needle location is available), however, preferably two or more tracking slices (basically all those for which images are produced) should be considered.

For example, a probability map can be used to identify a penetration point of the needle through the number of tracking slices imaged. The location of the needle in space can be determined based on the known locations of the tracking slices and the identified penetration points.

The needle-location algorithm is an AI algorithm (algorithm with artificial intelligence), in particular based on a regression method and estimates the needle location based on the recognized needle artifacts.

The position of the needle is preferably determined taking account of the artifact geometry (offset from the true position).

Location of a Monitoring Slice

Now, the location of a monitoring slice is calculated. This calculation is based on the identified location of the needle in the number of tracking slices. Since, in practice, the monitoring slice has a certain width, it generally surrounds the very thin needle. Therefore, the position and orientation information determined for the needle location in the tracking slice is used to adapt the position vector and normal vector of the monitoring slice.

For example, the penetration points of the needle through the tracking slices are identified. The position of the penetration points in the tracking slices and the known position of the tracking slices (from the location data) can be used to define at least one degree of freedom of the monitoring slice. A straight line through the penetration points defines a straight line that spans the plane which could only rotate about this straight line. The magnetic resonance tomography system can define the remaining degrees of freedom for this plane. This plane is then used to indicate the location of the monitoring slice. To define the monitoring slice in the space, it is, for example, possible to define the preferred direction for the alignment of the monitoring slice to enable optimal imaging of a contrast. However, it is also possible to select a direction with which the tracking slices are also aligned.

For example, it is also possible for the position of the needle point to be set as the position of the image center and for the normal vector of the monitoring slice to be obtained from the needle direction. The user can set a configuration to deal with ambiguities. For example, it is possible always to select the normal vector with the smallest angle to the normal vector of a transverse, sagittal or coronal slice.

Imaging a Monitoring Slice

The monitoring slice is now imaged in its calculated location. Depending upon the case in question, it may be preferable to image a single monitoring slice or even a plurality of monitoring slices. On the basis of the aforementioned identification of its location, the monitoring slice should be located and structured such that it includes the identified needle location.

The monitoring slice is particularly preferably imaged as a contrast, wherein theoretically it is possible for one single contrast to be imaged, although two or more contrasts are advantageous since they improve accuracy. Basically, the statements made in this respect with regard to the tracking slice apply. Hereinafter, monitoring slice in particular also includes the number of contrasts imaged.

It should be noted that, as soon as the needle is inserted into the patient, the degrees of freedom are significantly restricted. Therefore, following a successful determination of the position in the tracking slices, the needle should typically always be visible during the imaging (including with high-speed imaging) of the monitoring slice.

Displaying/Further Location Identification

The monitoring slice is now displayed, preferably in the form of a reconstruction of the imaging of a contrast, in particular together with a depiction of the identified needle location. Alternatively or additionally, a further identification of the location of the needle is performed, as described in more detail below.

An apparatus according to the disclosure for detecting a needle in magnetic-resonance images includes the following components:

A data interface configured to acquire location data provided on the location of a number of tracking slices. It is also used to output commands to produce images of a number of tracking slices in the form of magnetic-resonance images with the location data provided and for imaging a monitoring slice in its calculated location. Preferably, it is furthermore used to output data for displaying the monitoring slice.

An artifact-identifying unit configured to identify the picture elements in a tracking slice which depict needle artifacts, wherein the artifact-identifying unit includes a needle-artifact algorithm trained on needle artifacts. As stated above, the artifact-identifying unit is theoretically able to identify needle artifacts with a single needle-artifact algorithm in both the tracking slice and the monitoring slice, but it is preferable for each slice to use a specially trained needle-artifact algorithm.

A location-identifying unit configured to identify a needle location, wherein the location-identifying unit includes a needle-location algorithm trained on an estimation of the needle location. Here, the same statements made with respect to the needle-artifact algorithm apply: it is preferable for each slice to use a specially trained needle-location algorithm.

A location-calculating unit configured to calculate the location of a monitoring slice, which is structured to include the identified needle location.

A control facility according to the disclosure for controlling a magnetic resonance tomography system which is embodied to perform a method according to the disclosure and/or includes an apparatus according to the disclosure.

A magnetic resonance tomography system according to the disclosure includes a control facility according to the disclosure.

The majority of the aforementioned components of the apparatus or the control facility can be implemented entirely or partially in the form of software modules in a processor of a corresponding apparatus or control facility. An extensively software-based implementation has the advantage that it is also possible to retrofit apparatuses or control facilities used to date in a simple way by means of a software update in order to work in the manner according to the disclosure. In this respect, the object is also achieved by a corresponding computer program product with a computer program that can be loaded directly into a computing system or a memory device of a control facility of magnetic resonance tomography system with program segments for executing all the steps of the method according to the disclosure when the program is executed in the computing system or the control facility. In addition to the computer program, such a computer program product can optionally also include additional parts such as, for example, documentation and/or additional components and also hardware components, such as for example hardware keys (dongles etc.) for using the software.

Transportation to the computing system or to the control facility and/or storage on or in the computing system or the control facility can take place via a computer-readable medium, for example a memory stick, a hard disk or another kind of transportable or integrated data carrier on which the program segments of the computer program which can be read-in and executed by a computing system or a computing unit of the control facility are stored. To this end, the computing unit can, for example, include one or more interacting microprocessors or the like.

Further particularly advantageous embodiments and developments of the disclosure can be derived from the dependent claims and the following description, wherein the claims of one category of claims can be also developed in a similar way to the claims and passages of the description to create another category of claims and in particular individual features of different exemplary embodiments or variants can be combined to create new embodiments or variants.

A preferred method used for further identification of the location of the needle includes the following steps:

Identification of the picture elements in the monitoring slice (pixels in a contrast) which depict needle artifacts by means of a needle-artifact algorithm trained on needle artifacts (possibly especially in the monitoring slice). Therefore, the needle artifacts in the monitoring slice are segmented by means of a machine-learning algorithm. Here again, preferably a probability map is created as described above for the identification of needle artifacts in the tracking slice.

Identification of needle location by means of a needle location algorithm trained on an estimation of the needle location (possibly especially in the monitoring slice), based on the (segmented) needle artifacts in the monitoring slice (M). This in particular also takes place based on identified penetration points of the needle through the tracking slices.

Displaying the needle location, in particular in a depicted contrast of the monitoring slice, and/or performance of a further iteration for the identification of the needle location.

A preferred method for performing a further iteration for the identification of the needle location includes the following steps:

Calculating the location of a number of tracking slices (here preferably two or more) based on the identified needle location from the monitoring slice. Herein, these tracking slices are aligned orthogonally to the identified needle location (and obviously spaced apart from one another). Then, there is a calculation of location data in accordance with the calculated location of these tracking slices. This location data has a format corresponding to that of the data mentioned at the start and replaces said data for the next iteration.

Repetition of the steps of the method in the form of an iteration.

The steps of an iteration are substantially performed sequentially. First, the two tracking slices are imaged. However, it is possible to the start the determination of the position of the needle artifact in this slice immediately after the imaging of the first slice. Hence, this can take place in parallel to the imaging of the second tracking slice. However, the two tracking slices can in principle also be imaged interleaved in parallel. The positions identified from the tracking slices are used to align the monitoring slice. The imaging of the monitoring slice is started after the processing of the tracking slices. Following the conclusion of the imaging, the needle artifact is also localized in the monitoring slice. Here it is once again possible to work in parallel when imaging a plurality of monitoring slices. The needle location is calculated from the combined information from all three slices. This can then be used as the basis for calculating the slice positions of the tracking slices in the next iteration step.

In a preferred method, a simultaneous multislice imaging method (SMS-imaging method) is used for the imaging of the tracking slices and/or monitoring slices.

In a preferred method, for imaging a tracking slice and/or monitoring slice, two or more contrasts are imaged for each slice (and particularly preferably also evaluated for the identification of the needle location). In this context, preferably at least one white-marker contrast is imaged. As described above, more contrasts enable better localization of the needle position by the respective algorithm used.

In a preferred method, for the imaging of a plurality of contrasts, a real-time pulse sequence is modified in order to reduce the imaging time. As a result, only minimal additional time is required. Herein, preferably gradient moments of existing gradient objects are (slightly) changed in the slice selection direction and/or phase-encoding direction and/or in readout direction. For example, in a bSSFP sequence, gradients are balanced in order to maintain a bSSFP sequence.

In a preferred method, the location of the needle in the tracking slice and/or monitoring slice is additionally identified using imaging parameters, preferably using values for position vectors and normal vectors of the slices, the alignment of the slices relative to the BO field, field of view (FOV) or hardware parameters for gradient non-linearities.

In a preferred method, the identification of the picture elements in a tracking slice and/or monitoring slice (pixels in a contrast) which depict needle artifacts is performed by means of a deep-learning network. This deep-learning network preferably includes a U-net convolutional network for biomedical image segmentation and a convolutional recurrent neural network (convolutional RNN), wherein preferably the U-net convolutional network first generates from the contrasts of the respective slice a probability map that segments a needle artifact in the image and the segmentation is preferably subsequently improved with the convolutional recurrent neural network (in order to suppress unwanted artifacts).

With a preferred apparatus, the slice positioning is performed in a real-time system of a magnetic resonance tomography system that plays out radio-frequency pulses and gradient pulses and/or the position is determined in a reconstruction unit and preferably then sent to the real-time system that controls the RF pulses and gradient pulses.

A preferred apparatus includes the following special features:

The artifact-identifying unit is embodied by means of a needle-artifact algorithm trained on needle artifacts to identify the picture elements in the monitoring slice which depict needle artifacts. In this regard, reference is made to the preceding description which stated that the needle artifacts in the monitoring slice generally differ systematically from the needle artifacts in the tracking slice. Therefore, the artifact-identifying unit preferably includes two differently trained algorithms. Theoretically this can take place in physically separate modules but, because of their similarity, here they are grouped together under the term “artifact-identifying unit”.

The needle-location identifying unit is embodied by means of a needle-location algorithm trained on an estimation of the needle location to identify the needle location based on the needle artifacts in the monitoring slice. In this regard, reference is made to the preceding description which stated that the identification of the needle location in the monitoring slice can differ systematically from the identification of the needle location in the tracking slice. Therefore, the needle-location identifying unit preferably includes two differently trained algorithms. Theoretically, this can take place in physically separate modules, but, because of their similarity, here they are grouped together under the term “needle-location identifying unit”.

The data interface is preferably configured to output data for displaying the needle location in the monitoring slice.

The slice-location calculating unit is preferably configured to calculate the location of a number of tracking slices based on the identified needle location from the monitoring slice, wherein the number of tracking slices is aligned orthogonally to the identified needle location. Furthermore, the slice-location calculating unit is preferably configured to create the relevant location data relating to the location of this number of tracking slices (and make it available for further iteration). Theoretically, these calculations can also take place in physically separate modules but, because of their similarity, here they are grouped together under the term “slice-location calculating unit”.

### BRIEF DESCRIPTION OF THE DRAWINGS

The disclosure is described again in more detail below with reference to the appended figures and with reference to exemplary embodiments. Herein, the same components are given identical reference characters in the different figures. The figures are generally not true to scale and show:

FIG. 1 a schematic depiction of a magnetic resonance tomography system according to an exemplary embodiment of the disclosure;

FIG. 2 a sketch depicting the slices required for the method;

FIG. 3 a flowchart for a possible sequence of a method according to the disclosure; and

FIG. 4 a preferred AI network.

The figures only show elements that are essential for the disclosure or helpful for understanding the disclosure.

### DETAILED DESCRIPTION

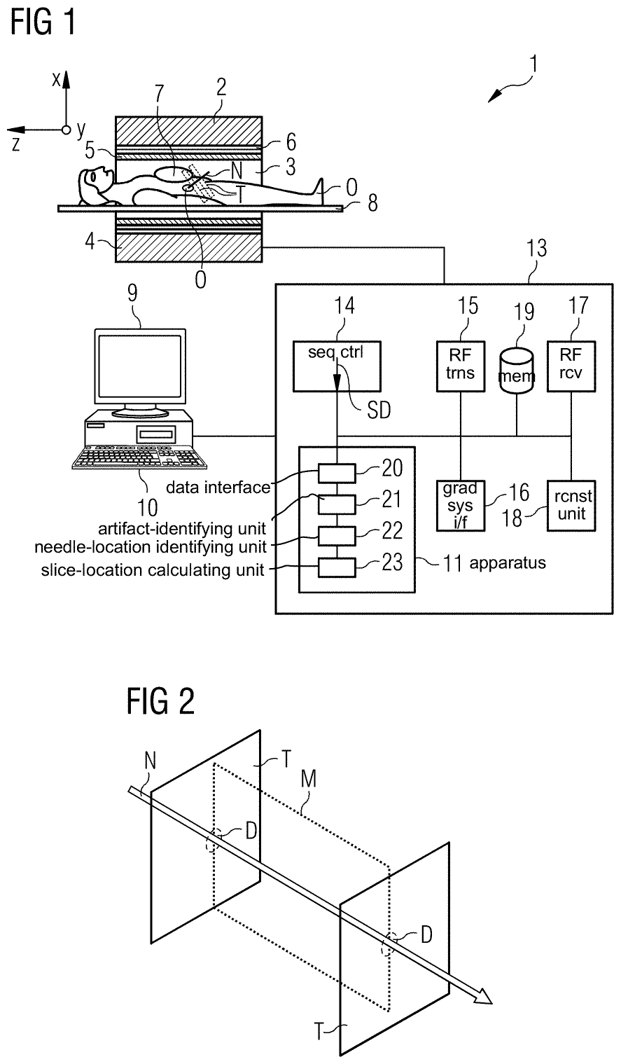

FIG. 1 is a rough schematic depiction of a magnetic resonance tomography system 1. It includes on the one hand the actual magnetic resonance scanner 2 with an examination space 3 or patient tunnel in which a patient or test subject in whose body the examination object O and a needle N for a medical intervention is located is positioned on a couch 8.

The magnetic resonance scanner 2 is equipped in the usual manner with a basic field magnetic system 4, a gradient system 6 together with an RF transmitting antenna system 5 and an RF receiving antenna system 7. In the exemplary embodiment shown, the RF transmitting antenna system 5 is a whole-body coil permanently installed in the magnetic resonance scanner 2, whereas the RF receiving antenna system 7 consists of local coils to be arranged on the patient or test subject (here this is symbolized by only one single local coil). However, in principle, the whole-body coil can also be used as the RF receiving antenna system and the local coils as the RF transmitting antenna system as long as these coils can each be switched to different operating modes. Here, the basic field magnetic system 4 is embodied in the normal manner in that it generates a basic magnetic field in the longitudinal direction of the patient, i.e. along the longitudinal axis of the magnetic resonance scanner 2 extending in the z direction. The gradient system 6 includes in the usual manner individually controllable gradient coils to enable gradients to be switched in the x, y or z direction independently of one another. The magnetic resonance scanner 2 also contains shim coils (not shown) which can be embodied in the usual manner.

The magnetic resonance tomography system 1 shown in FIG. 1 is a whole-body system with a patient tunnel into which a patient can be completely introduced. However, in principle, the disclosure can also be used on other magnetic resonance tomography systems, for example with a laterally open, C-shaped housing. It is only essential for it to be possible to produce corresponding images of the examination object O.

The magnetic resonance tomography system 1 is configured to produce images of different slices. Imaging of individual slices is known to the person skilled in the art. To this end, generally gradient pulses are applied during a scan at exactly specified positions in time and with a precisely defined course over time. As a result, the examination object O is scanned in individual slices together with the needle N and the associated k-space. Here, two tracking slices T are shown which are arranged orthogonally to the needle N.

The magnetic resonance tomography system 1 furthermore comprises a central control facility 13, which is used to control the MR system 1. This central control facility 13 includes a sequence control unit 14. This controls the sequence of radio-frequency pulses (RF pulses) and gradient pulses as a function of a selected pulse sequence PS or a sequence of a plurality of pulse sequences for producing images of a plurality of slices in a volume region of interest of the examination object within a scanning session. Such a pulse sequence PS can, for example, be specified and parameterized as part of a scanning or control protocol. Usually, different control protocols for different scans or scanning sessions are stored in a memory 19 and can be selected by a user (and possibly amended if required) and then used to perform the scan.

To output the individual RF pulses of a pulse sequence PS, the central control facility 13 comprises a radio-frequency transmission facility 15, which generates and amplifies the RF pulses and feeds them via a suitable interface (not shown in detail) into the RF transmitting antenna system 5. To control the gradient coils of the gradient system 6 in order to switch the gradient pulses appropriately in accordance with the specified pulse sequence PS, the control facility 13 comprises a gradient system interface 16. Gradient pulses could be applied via this gradient system interface 16. The sequence control unit 14 communicates in a suitable manner, for example by transmitting sequence control data SD, with the radio-frequency transmission facility 15 and the gradient system interface 16 in order to execute the pulse sequence PS.

The control facility 13 also comprises a radio-frequency receiving facility 17 (which also communicates in a suitable manner with the sequence control unit 14) in order to receive magnetic resonance signals in a coordinated manner by means of the RF receiving antenna system 7 within the readout window specified by the pulse sequence PS and thus acquire the raw data.

Here, a reconstruction unit 18 accepts the acquired raw data and reconstructs magnetic resonance image data therefrom. This reconstruction is also generally performed on the basis of parameters that can be specified in the respective scan or control protocol P. This image data can then be stored in a memory 19, for example.

The manner by which suitable raw data can be acquired by the irradiation of RF pulses and the switching of gradient pulses and MR images or parameter maps reconstructed therefrom is known in principle to the person skilled in the art and will not, therefore, be explained in any more detail here.

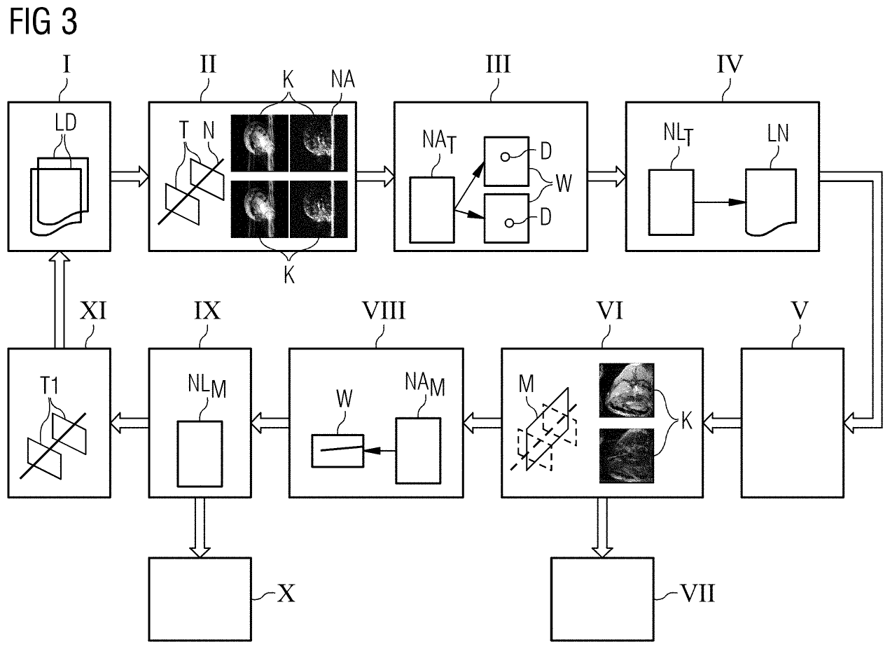

To carry out the method according to the disclosure (see for example FIG. 3), the control facility 13 includes an apparatus 11 for detecting a needle N in magnetic-resonance images K. This apparatus communicates with the other components, in particular with the image reconstruction unit 16, the sequence control unit 14, or the gradient system interface 16, a memory 19 and preferably also a display unit 9 via a data interface 20.

This data interface 20 is configured to acquire location data LD provided on the location of a number of tracking slices T, which here it receives from the memory 19 or the terminal 11. The data interface 20 is furthermore used to output commands for imaging

a) a number of tracking slices T in the form of magnetic-resonance images K with the location data LD provided,
b) a monitoring slice M in its calculated location,

and preferably furthermore to output data for displaying the monitoring slice M and/or further identification of the needle location LN.

An artifact-identifying unit 21 is used to identify the picture elements in a tracking slice T that depict elements of the needle N, wherein the artifact-identifying unit 21 includes a needle-artifact algorithm NA_{T} trained on needle artifacts in the tracking slice T. It can additionally comprise a needle-artifact algorithm NA_{M} trained on needle artifacts in a monitoring slice M.

A needle-location identifying unit 22 is used to identify a needle location LN, wherein the needle-location identifying unit 22 comprises a needle-location algorithm NL_{T} trained on an estimation of the needle location LN in the tracking slice T. It can additionally include a needle-location algorithm NL_{M} trained on an estimation of the needle location LN in a monitoring slice M.

A slice-location calculating unit 23 is used to calculate the location of a slice, here at least one monitoring slice M, which is structured such that it includes the identified needle location LN.

The central control facility 13 can be operated via a terminal 10 with an input unit and a display unit 9 via which the entire magnetic resonance tomography system 1 can hence be operated by one operator. Magnetic resonance tomography images can also be displayed on the display unit 9 and scans can also be planned and started by means of the terminal.

The magnetic resonance tomography system 1 according to the disclosure and in particular the control facility 13 can moreover comprise a multiplicity of further components, which are not shown individually here but are usually present on such systems, such as, for example a network interface in order to connect the entire system to a network and to enable the exchange of raw data and/or image data or parameter maps and also further data, such as, for example, patient-related data or control protocols.

The manner by which suitable raw data can be acquired by the irradiation of RF pulses and the generation of gradient fields and magnetic resonance tomography images reconstructed therefrom is known in principle to the person skilled in the art and will not be explained in any more detail here. Similarly, a wide variety of scan sequences, such as, for example, EPI scan sequences or other scan sequences for the generation of diffusion-weighted images are known in principle to the person skilled in the art.

FIG. 2 is a perspective sketch depicting the slices required for the method. A needle N travels from left to right, wherein the tip of the needle N is indicated as an arrow head. Two tracking slices T are aligned orthogonally to the needle, wherein the needle passes through the tracking slices T at the penetration points D. These penetration points D are connected by a monitoring slice M. In the depiction, the monitoring slice M only includes the space between the two tracking slices T. This does not necessarily have to be the case since an MRI system is basically able to accommodate slices of any size (within the limits imposed by the system). Therefore, the tip of the needle N could also lie within the monitoring slice M and the endpoint of the needle N determined very accurately in the monitoring slice M in the case of needle-artifact determination and location determination.

FIG. 3 is a block diagram depicting the exemplary course of a method according to the disclosure for detecting a needle in magnetic-resonance images.

Step I entails the provision of location data LD on the location of two tracking slices T. This location data LD is initially estimations of the needle location and can, for example, be obtained by manually inputting or from data on the type of intervention.

Step II entails the imaging of the two tracking slices T in the form of two contrasts in each case with a magnetic resonance tomography system 1 as is depicted, for example, in FIG. 1. A needle artifact NA is indicated in one of the contrasts K. The other contrasts K also contain needle artifacts NA, which, for purposes of simplicity, are not additionally identified by reference characters. Herein, the tracking slices T are imaged with the location data LD provided.

Step III entails the identification of the pixels in a tracking slice T which depict needle artifacts NA by means of a needle-artifact algorithm NA_{T} trained on needle artifacts NA. Herein, two probability maps W are obtained in which the penetration points D can be recognized.

Step IV entails the identification of a needle location LN by means of a needle-location algorithm NL_{T} trained on an estimation of the needle location LN from the identified pixels which depict needle artifacts NA in the two tracking slices T.

Step V entails the calculation of the location of a monitoring slice M which is structured such that it includes the identified needle location LN.

Step VI entails the imaging of a monitoring slice M in its calculated location.

Step VII entails displaying the monitoring slice.

Step VIII entails the identification of the pixels in the monitoring slice M, which depict needle artifacts NA by means of a further needle-artifact algorithm NA_{M} trained on needle artifacts NA in the monitoring slice. Since the needle artifacts NA are generally structured differently in the monitoring slice than in the tracking slice, although the needle-artifact algorithm NA_{M} generally operates here in accordance with the same basic principle as that used above with the tracking slice, it was trained differently. Here once again, a probability map is created.

Step IX entails the identification of the needle location LN by means of a needle-location algorithm NL_{M} trained on an estimation of the needle location based on the segmented needle artifacts NA in the monitoring slice M.

Step X entails displaying the needle location LN in the monitoring slice M.

Step XI entails the calculation of the location of a further two tracking slices T1 based on the identified needle location LN from monitoring slice M, wherein the number of the further two tracking slices T1 are aligned orthogonally to the identified location of the needle.

This calculated location is used as location data LD for a repetition of the method in the context of an iteration step. This iteration can be performed multiple times, for example until a limit value for the difference in the values for the needle location between two successive iterations is fallen below.

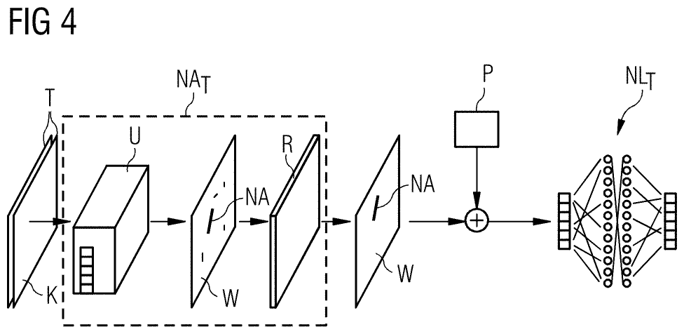

FIG. 4 shows a preferred AI network for the determination of needle artifacts NA in slices, which in this example are two tracking slices T. The left-hand side shows two contrasts K, which were produced in the context of the imaging of the two tracking slices T. This first entails the identification of the pixels in a contrast K which depict elements of the needle N by means of a U-net convolutional network U for biomedical image segmentation. This U-net convolutional network U generates from the respective contrast (i.e. from the respective tracking slice T) a probability map W that segments the needle artifact NA in the image. Here, it may be seen that, in addition to the needle artifact NA, the probability map W contains further striated structures. These depict errors, which are eliminated in a further step by means of a convolutional recurrent neural network R. This improves segmentation. These two networks together produce the needle-artifact algorithm NA_{T}.

In a subsequent step, possibly with additional scan parameters P (for example from a DICOM header), the location of the needle is identified with a needle-location algorithm NL_{T} depicting a further deep-learning network, for example a convolutional network).

Finally, reference is made once again to the fact that the method described in detail above and the magnetic resonance tomography system 1 are exemplary embodiments only and can be modified by the person skilled in the art in a variety of ways without leaving the scope of the disclosure. Furthermore, the use of the indefinite articles “a” or “an” does not exclude the possibility that the features in question may also be present on a multiple basis. Similarly, the terms “unit” and “module” do not preclude the possibility that the components in question consist of a plurality of interacting partial components, which could also be spatially distributed.

1. A method for detecting a needle in magnetic-resonance images, wherein a needle artifact and/or a needle location is identified by means of algorithms based on artificial intelligence within the context of machine learning; the method comprising:
providing location data on the location of a number of tracking slices;
imaging the number of tracking slices in the form of magnetic-resonance images with the location data;
identifying picture elements in a tracking slice depicting needle artifacts by means of a needle-artifact algorithm trained on needle artifacts;
identifying a needle location by means of a needle-location algorithm trained on an estimation of the needle location from the identified picture elements in a tracking slice depicting needle artifacts;
calculating a location of a monitoring slice based on the identified needle location;
imaging the monitoring slice in its calculated location; and
displaying the monitoring slice or further identifying the needle location.

2. The method as claimed in claim 1,
wherein the further identifying the needle location comprises:
identifying the picture elements in the monitoring slice depicting needle artifacts by means of a needle-artifact algorithm trained on needle artifacts;
identifying the needle location by means of a needle-location algorithm trained on an estimation of the needle location based on the needle artifacts in the monitoring slice; and
displaying the needle location or performing a further iteration to identify the needle location.

3. The method as claimed in claim 2, wherein the performing the further iteration to identify the needle location comprises:
calculating the location of a number of tracking slices based on the identified needle location from the monitoring slice, wherein the number of tracking slices are aligned orthogonally to the identified needle location;
calculating location data in accordance with the calculated location of this number of tracking slices; and
repeating the calculating steps of the performing the further iteration in the form of an iteration.

4. The method as claimed in claim 2, further comprising using a simultaneous multislice imaging method for the imaging of the tracking slices or monitoring slices.

5. The method as claimed in claim 2, wherein the imaging the tracking slice or monitoring slice comprises imaging two or more contrasts for each slice, wherein at least one white marker contrast is imaged.

6. The method as claimed in claim 5,
wherein the imaging of the contrasts comprises modifying a real-time pulse sequence in order to reduce the imaging time, and
the method further comprises changing gradient moments of existing gradient objects in a slice-selection direction, a phase-encoding direction, or a readout direction.

7. The method as claimed in claim 1, further comprising:
additionally identifying the needle location, in a tracking slice or monitoring slice, using imaging parameters, values for position vectors and normal vectors of the slices, the alignment of the slices relative to the BO field, field of view, or hardware parameters for gradient non-linearities.

8. The method as claimed in claim 2, further comprising:
performing the identifying of the picture elements, in a tracking slice or monitoring slice, which depict needle artifacts by means of a deep-learning network; and
subsequently improving, with a convolutional recurrent neural network, generating a U-net convolutional network for biomedical image segmentation from the respective slice a probability map that segments the needle artifact in the image and the segmentation.

9. (canceled)

10. (canceled)

11. (canceled)

12. A control facility for controlling a magnetic resonance tomography system, which is embodied to perform a method as claimed in claim 1.

13. (canceled)

14. (canceled)

15. A non-transitory computer program product with a computer program, which can be loaded directly into a storage facility of a control facility of a magnetic resonance tomography system, with program segments for executing the steps of the method as claimed in claim 1 when the computer program is executed in the control facility of the magnetic resonance tomography system.

16. A non-transitory computer-readable medium on which program segments that can be read and executed by a computer are stored in order to execute the steps of the method as claimed in claim 1, when the program segments are executed by the computer.
